(19) United States
(12) Patent Application Publication
(10) Pub. No.: US 2025/0367350 A1
(43) Pub. Date: Dec. 4, 2025

(54) CROSSLINKED HYDROGEL COATED MEDICAL GRAFTS

(71) Applicant: COOK BIOTECH INCORPORATED, WEST LAFAYETTE, IN (US)

(72) Inventors: SYDNEY E. HOLLINGSHEAD, LAFAYETTE, IN (US); TYLER A. NOVAK, LAFAYETTE, IN (US)

(73) Assignee: COOK BIOTECH INCORPORATED, WEST LAFAYETTE, IN (US)

(21) Appl. No.: 19/227,780

(22) Filed: Jun. 4, 2025

Related U.S. Application Data
(60) Provisional application No. 63/655,664, filed on Jun. 4, 2024.

(51) Int. Cl. A61L 27/52; A61L 27/22

(52) U.S. Cl. CPC A61L 27/52; A61L 27/22

(57) ABSTRACT

In certain embodiments, the present disclosure provides medical graft devices having covalently crosslinked matrix protein hydrogel coatings. In some forms, such coatings are configured to reduce the formation of adhesions upon implantation.

### BACKGROUND

The present invention resides generally in the field of medical compositions and in particular aspects to medical compositions that incorporate extracellular matrix materials.

As further background, hernia repair frequently results in soft tissue adhesion in the repair site. Adhesions can be caused by drying of tissue during surgery, irritation or abrasion by tools and equipment, or friction against implanted grafts, among other causes. Adhesions often occur on the surface of implanted hernia mesh grafts, where internal organs rub against the device.

Soft tissue adhesions significantly complicate future surgeries in the same area by obscuring organ and tissue structures. These complications can include extended surgery time, increased difficulty of the surgery, or potentially refusal of the surgeon to complete the surgery. Adhesions are also hypothesized to correlate with abdominal pain.

A need remains for improved or alternative medical compositions and products that can be used in a wide variety of medical applications, in some cases to minimize adhesions of implanted materials with surrounding soft tissues. The present invention provides such medical compositions and products, as well as methods for preparing and using the same.

### SUMMARY

In certain aspects, the present disclosure pertains to medical graft devices, in particular to medical graft devices having a surface coating. In some forms, the present disclosure provides medical graft devices having a lubricious coating configured to reduce the formation of adhesions upon implantation. In accordance with certain embodiments, the present disclosure provides an implantable medical graft comprising a substrate contained within a sterile package, the substrate comprising a collagenous extracellular matrix material, and a coating disposed on a first face of the substrate. The coating comprises a composition of cross-linked matrix proteins having diphenolic crosslinks between individual matrix proteins within the coating material, and diphenolic crosslinks between matrix proteins and the substrate. In accordance with some forms, the coating may include hyaluronic acid, sodium hyaluronate, chondroitin sulfate, and/or one or more added sugars. In some forms, the collagenous extracellular matrix material comprises submucosa, dura mater, pericardium, basement membrane, or dermal collagen. In certain embodiments, a second face of the substrate is free of the coating. In certain embodiments, the coating is disposed on a second face of the substrate. In accordance with certain embodiments, the medical graft is lyophilized. In some forms, the medical graft comprises one or more openings in the medical graft material extending through the coating layer(s) and the substrate. In certain embodiments, the coating covers substantially all of the face of the substrate upon which it is disposed. In accordance with some forms, one or more faces of the substrate comprises coated portions and uncoated portions.

The present disclosure also provides methods of making an implantable medical graft. In certain embodiments such methods comprise crosslinking a collagenous extracellular matrix substrate material, the substrate material having a coating disposed on a first face of the substrate material, wherein said crosslinking is effective to form diphenolic cross-links between matrix proteins within the coating and diphenolic crosslinks between matrix proteins of the coating and the substrate to form a crosslinked medical graft, and sealing the crosslinked medical graft within a sterile package. In accordance with some forms, the coating may include hyaluronic acid, sodium hyaluronate, chondroitin sulfate, and/or one or more added sugars. In some forms, the collagenous extracellular matrix material comprises submucosa, dura mater, pericardium, basement membrane, or dermal collagen. In certain embodiments, a second face of the substrate is free of the coating. In certain embodiments, the coating is disposed on a second face of the substrate. In accordance with certain embodiments, the medical graft is lyophilized. In some forms, the method comprises forming one or more openings in the medical graft material extending through the coating layer(s) and the substrate. In certain embodiments, the coating covers substantially all of the face of the substrate upon which it is disposed. In accordance with some forms, one or more faces of the substrate comprises coated portions and uncoated portions.

The present disclosure provides methods of treating a patient. In certain embodiments, such methods comprise implanting a crosslinked medical graft comprising a collagenous extracellular matrix material substrate and a coating disposed on at least a first face of the substrate, the coating comprising a cross-linked matrix protein having diphenolic cross-links between matrix proteins within the coating and diphenolic cross-links between the matrix proteins and the substrate. In some forms, the method includes the step of removing the crosslinked medical graft material from a sterile package.

Additional embodiments, as well as features and advantages of embodiments of the invention, will be apparent from the description herein.

### BRIEF DESCRIPTION OF THE DRAWINGS

FIG. 1 is a perspective view of one embodiment of a medical graft material of the present disclosure.

FIG. 2 is a top-down view of one embodiment of a medical graft material of the present disclosure.

FIG. 3a is a side view of one embodiment of a medical graft material of the present disclosure.

FIG. 3b is a side view of one embodiment of a medical graft material of the present disclosure.

FIG. 4a is a top-down view of one embodiment of a medical graft material of the present disclosure.

FIG. 4b is a top-down view of one embodiment of a medical graft material of the present disclosure.

FIG. 5a is a perspective view of one embodiment of a medical graft material of the present disclosure.

FIG. 5b is a side view of one embodiment of a medical graft material of the present disclosure.

FIG. 6 is a perspective view of one embodiment of a medical graft material of the present disclosure.

FIG. 7 illustrates the results of the experiment detailed in Example 3, the extent of modification with varying Bolton Hunter reagent concentrations.

### DESCRIPTION OF THE SELECTED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated graft, and further applications of the principles of the disclosure as illustrated therein are herein contemplated as would normally occur to one skilled in the art to which the disclosure relates.

As disclosed above, aspects of the present disclosure relate to novel methods and materials for coating a graft material. In certain aspects, the disclosure relates to implantable graft materials having one or more coated surfaces. As will be discussed herein, it has been found that medical graft devices as disclosed herein reduce the formation of tissue adhesions upon implantation. In accordance with some forms, the coatings disclosed herein include one or more polymers containing phenolic groups. In certain embodiments the coating comprises a diphenolic crosslinked hydrogel material.

In some forms, the present disclosure provides a medical implant graft comprising a substrate material. In certain embodiments, the substrate material comprises a remodelable material. Particular advantage can be provided by including a remodelable collagenous material. Such remodelable collagenous materials can be provided, for example, by collagenous materials isolated from a suitable tissue source from a warm-blooded vertebrate, and especially a mammal. Reconstituted or naturally-derived collagenous materials can be used in the present invention. Such materials that are at least bioresorbable will provide advantage in the present invention, with materials that are bioremodelable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth.

Suitable bioremodelable materials can be provided by collagenous extracellular matrix materials (ECMs) possessing biotropic properties, including in certain forms angiogenic collagenous extracellular matrix materials. For example, ECMs include materials such as submucosa, renal capsule membrane, dermal collagen, dura mater, pericardium, fascia lata, serosa, peritoneum or basement membrane layers, including liver basement membrane. Suitable submucosa-containing materials for these purposes include, for instance, materials that include intestinal submucosa, including small intestinal submucosa, stomach submucosa, urinary bladder submucosa, and uterine submucosa. These identified submucosa or other layers can occur in the ECM material alone, or in combination with other materials such as those derived from one or more adjacent layers in the source tissue.

The submucosa-containing ECM can be derived from any suitable organ or other biological structure, including for example submucosa derived from the alimentary, respiratory, intestinal, urinary or genital tracts of warm-blooded vertebrates. Submucosa-containing materials useful in the present invention can be obtained by harvesting such tissue sources and delaminating the submucosa (alone or combined with other materials) from smooth muscle layers, mucosal layers, and/or other layers occurring in the tissue source. For additional information as to submucosal materials useful in the present invention, and its isolation and treatment, reference can be made, for example, to U.S. Pat. Nos. 4,902,508, 5,554,389, 5,993,844, 6,206,931, and 6,099,567.

When a submucosal or other ECM material having differing characteristic sides is used in combination with a coating, the coating can be oriented upon the medical graft on a specified side. For example, in the case of small intestinal submucosa, the coating may be oriented in any manner as described herein, on either the luminal or abluminal side of the small intestinal submucosa.

As prepared, the submucosal material and any other ECM used may optionally retain growth factors or other bioactive components native to the source tissue. For example, the submucosal or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a native bioactive component that induces, directly or indirectly, a cellular response such as a change in cell morphology, proliferation, growth, protein or gene expression.

Submucosal or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

Further, in addition or as an alternative to the inclusion of native bioactive components, non-native bioactive components such as those synthetically produced by recombinant technology or other methods, may be incorporated into the submucosal or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient. Alternatively, or additionally, a non-native bioactive component can be included in the coating material of the medical product. When included in the coating, the non-native bioactive component can be added at any point during preparation of the medical product, including being mixed with one or all of the coating components prior to application of the coating to a surface of a layer of a medical material or, alternatively, after the coating is formed, applied, or cross-linked.

A non-native bioactive component can be applied to a submucosal or other ECM tissue by any suitable means. Suitable means include, for example, spraying, impregnating, dipping, etc. The non-native bioactive component can be applied to the ECM tissue either before or after the coating is applied to the material, or both. Similarly, if other chemical or biological components are included in the ECM tissue, the non-native bioactive component can be applied either before, in conjunction with, or after these other components.

Submucosal or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosal or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosal tissue used in the present invention.

With respect to the above, a medical material can be provided in any suitable form prior to application of a coating thereto. Suitable forms include, for example, as one or more sheets or layers, as a foam, or as a sponge. The form used will typically depend on a variety of factors including, but not limited to, the end use of the medical product and the type of material used (e.g., synthetic or biological).

When used in the invention, ECM materials may be essentially free of additional, non-native crosslinking, or may contain additional crosslinking prior to application of the coating material. Such additional crosslinking may be achieved by photo-crosslinking techniques, by chemical crosslinkers, or by protein crosslinking induced by dehydration or other means. However, because certain crosslinking techniques, certain crosslinking agents, and/or certain degrees of crosslinking can destroy the remodelable properties of a remodelable material, where preservation of remodelable properties is desired, any crosslinking of the remodelable ECM material can be performed to an extent or in a fashion that allows the material to retain at least a portion of its remodelable properties. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

In embodiments of the invention where a medical material is provided in sheet form, the medical material will have a thickness in the range of about 50 to about 1000 microns, more preferably about 100 to 600 microns, and most preferably about 100 to about 350 microns. If necessary or desired, a multilaminate medical product can be used. For example, a plurality of (i.e. two or more) layers of a biocompatible material, for example submucosa-containing or other ECM material, can be bonded together to form a multilaminate structure. Illustratively, two, three, four, five, six, seven, or eight or more layers of a biocompatible material can be bonded together to provide a multilaminate bolster material. In certain embodiments, two to six collagenous, submucosa-containing layers isolated from intestinal tissue of a warm-blooded vertebrate, particularly small intestinal tissue, are bonded together to provide the staple bolster material. Porcine-derived small intestinal tissue is preferred for this purpose. The layers of collagenous tissue can be bonded together in any suitable fashion, including dehydrothermal bonding under heated, non-heated or lyophilization conditions, stitching, using adhesives as described herein, glues or other bonding agents, crosslinking with chemical agents or radiation (including UV radiation), or any combination of these with each other or other suitable methods.

Multilaminate structures when used in the invention can include a plurality of ECM material layers bonded together, a plurality of non-ECM material layers bonded together, or a combination of one or more ECM material layers and one or more non-ECM material layers bonded together. Illustratively, two or more ECM segments can be fused or bonded together using a bonding technique, such as chemical cross-linking or vacuum pressing during dehydrating conditions. An adhesive, glue or other bonding agent may also be used in achieving a bond between material layers. Suitable bonding agents may include, for example, collagen gels or pastes, gelatin, or other agents including reactive monomers or polymers, for example cyanoacrylate adhesives. A combination of one or more of these with dehydration-induced bonding may also be used to bond ECM material layers to one another. Additionally, or alternatively, the coating described herein may be dehydrated. In accordance with some forms, the coating is applied to the substrate material forming a graft material, and the coated graft material is dehydrated forming a dehydrated medical graft.

A variety of dehydration-induced bonding methods can be used to fuse ECM portions together, and or dehydrate coating and/or medical graft material. In one preferred embodiment, multiple layers of ECM material and/or coating layers are compressed under dehydrating conditions. The term “dehydrating conditions” can include any mechanical or environmental condition which promotes or induces the removal of water from the ECM material. To promote dehydration of the compressed ECM material, at least one of the two surfaces compressing the matrix structure can be water permeable. Dehydration of the graft material can optionally be further enhanced by applying blotting material, heating the matrix structure or blowing air, or other inert gas, across the exterior of the compressing surfaces. One particularly useful method of dehydration bonding ECM materials is lyophilization, e.g. freeze-drying or evaporative cooling conditions. In some forms, lyophilization is used to dehydrate a coating as described herein, preferably a medical graft including a coating layer is lyophilized.

Another method of dehydration bonding comprises pulling a vacuum on the assembly while simultaneously pressing the assembly together. This method is known as vacuum pressing. During vacuum pressing, dehydration of the ECM materials in forced contact with one another effectively bonds the materials to one another, even in the absence of other agents for achieving a bond, although such agents can be used while also taking advantage at least in part of the dehydration-induced bonding. With sufficient compression and dehydration, the ECM materials can be caused to form a generally unitary ECM structure.

It is sometimes advantageous to perform drying operations under relatively mild temperature exposure conditions that minimize deleterious effects upon the ECM materials of the invention, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating-at about room temperature (about 25° C.) or with cooling. Relatively low temperature conditions also, of course, include lyophilization conditions.

Turning now to a discussion of coating materials, the coating can be applied to at least a portion of a surface of a medical material by any suitable means. Suitable means include, for example, brushing, spraying, dipping, etc. Alternatively, a dried coating film can be separately prepared, and then attached to the medical material, e.g. by partial wetting of one side and bonding of that side to the medical material, optionally followed by re-drying. Typically, a substantial portion of a surface of a medical material is coated. By “substantial portion” is meant that at least about 75% of a specified surface (e.g. one side or the other of a sheet in certain circumstances) of a medical material is coated. The coating can be applied to the medical material at the point of use, or in a pre-applied configuration.

In accordance with certain embodiments, the present disclosure provides a coating comprising a cross-linked matrix protein. In some forms, the cross-linked matrix protein includes individual proteins having diferuloyl cross-links between individual polymers and/or cross-links between polymers of the coating material and the substrate. Such diferuloyl crosslinks are, more generally, crosslinks between phenolic groups of separate polymer molecules (“diphenolic crosslinks”). Where the coating material also includes a polymer containing phenolic groups other than the phenol-enriched synthetic polymer, the curing process may also form diphenolic crosslinks between the phenol-enriched synthetic polymer molecules and molecules of the other polymer containing phenolic groups, as well as between separate polymer molecules of the other polymer containing phenolic groups. As used herein the term “phenolic group” refers to a phenyl ring having a hydroxyl group directly attached to a carbon atom of the phenyl ring. The phenyl ring can optionally have other functional groups attached thereto. For example, a feruloyl group (which has a 4-hydroxy-3-methoxyphenyl group) is a phenolic group as described herein.

In certain embodiments, a coating as described herein is formed by irradiating a coating solution comprising a polymer such as matrix protein, a photoactivatable catalyst such as a photoactivatable metal-ligand complex or riboflavin, and an electron acceptor thereby initiating a cross-linking reaction to form a coating comprising the cross-linked matrix protein. The crosslinked material comprises a crosslinked polymeric matrix including the polymer and having covalent feruloyl-feruloyl crosslinks between polymer molecules. The cured (crosslinked) material can further include a photoactivatable catalyst such as a photoactivatable metal-ligand complex and/or an electron acceptor, and can include a reaction product obtained by photocuring a photocurable adhesive composition including a phenol-enhanced synthetic polymer, the photoactivatable catalyst, the electron acceptor, and a liquid medium. In some forms, the irradiating is conducted prior to implantation of the medical graft. For example, in certain embodiments the irradiating is conducted prior to placing the cross-linked graft into a sterile medical package. However, in certain embodiments the graft may be irradiated shortly before implantation. In such cases, a coating solution may be applied to a substrate and irradiated prior to implantation.

When as discussed herein, a coating is formed on a graft material, the graft material can include an extracellular matrix sheet material, and/or can have a porous matrix formed by a network of fibers, the porous matrix having pores formed between the fibers of the network. When the graft material has a porous matrix, such coating as described herein can include a first portion infiltrating pores of the porous matrix and a second portion external of the porous matrix.

As used herein the term “matrix protein” refers to isolated and purified extracellular matrix proteins. Suitable matrix proteins for use in the coating may be selected from, but not limited to the group consisting of: fibrinogen, fibrin, collagen, keratin, gelatin, fibronectin, serum albumin, elastin, beta-lactoglobulin, glycinin, glutens, gliadins, resilin and/or laminin, or admixtures thereof. Matrix proteins may be isolated from human or animal sources or can be synthetically produced for instance using recombinant techniques. In some forms, the protein may be denatured to encourage the formation of phenolic cross-links upon photocuring and/or may be a phenol enriched protein (e.g. modified to increase the number of tyrosine groups). Denaturation of a protein may be accomplished by raising or lowering the pH of a solution containing the matrix protein, decreasing or increasing the ionic strength of a solution containing the matrix protein, hydrolysis, or in other ways known to a person skilled in the art. Chemical modification to form a phenol enriched polymer material (e.g. including chemically added tyrosine groups) may be achieved by any suitable method. In some forms, such chemical modification may include the modification of amino acid side chains to include aromatic moieties such as the phenolic moiety present in tyrosine. By way of example primary amines such as the lysine residues in a protein may be modified using the known Bolton Hunter reagent (N-succinimidyl-3-[4-hydroxyphenyl]propionate) or the known water-soluble Bolton Hunter reagent (sulfosuccinimidyl-3-[4-hydroxyphenyl]propionate). In certain forms, the coating will include a mixture of an amount of a protein (especially collagen, gelatin or a collagen peptide composition) with an amount of the corresponding phenol enriched protein. For example, the photocured coating material may include the parent (unmodified) protein and the corresponding phenol enriched protein in a dry weight ratio in the range of about 1:10 to about 10:1, or about 1:5 to about 5:1, and in some forms about 5:1 to about 2:1. In certain embodiments, such ratios are used when the parent (unmodified) protein is collagen, gelatin, or a collagen peptide composition.

While not wishing to be bound by theory, it is believed that the mechanism involves irradiation of the photoactivatable catalyst, for example a photoactivatable metal-ligand complex to induce an excited state, followed by transfer of an electron from the metal to an electron acceptor. The oxidized metal then extracts an electron from a side chain such as a tyrosine side chain in the protein to produce, a tyrosyl radical that reacts immediately with a nearby tyrosine to form a dityrosine bond. A direct cross-link (without any bridging moiety) is created quickly in this photo-initiated chemical reaction, without the need for introduction of a primer layer and without the generation of potentially detrimental species such as singlet oxygen, superoxide and hydroxyl radicals. The term “photoactivatable metal-ligand complex” as used herein means a metal-ligand complex in which the metal can enter an excited state when irradiated such that it can donate an electron to an electron acceptor in order to move to a higher oxidation state and thereafter extract an electron from a side chain of an amino acid residue of a matrix protein to produce a free radical without reliance upon the formation of singlet oxygen. Suitable metals include but are not limited to Ru(II), Pd(II), Cu(II), Ni(II), Mn(II) and Fe(III) in the form of a complex which can absorb light in the visible region, for example, an Ru(II) bipyridyl complex, a Pd(II) porphyrin complex, a sulfonatophenyl Mn(II) complex or a Fe(III) protoporphyrin complex, more particularly, an Ru(II) bispyridyl complex or a Pd(II) porphyrin, in particular, an Ru(II) (bpy)₃ complex such as [Ru(II) (bpy)₃]Cl₂. Efficient cross-linking occurs in the presence of an electron acceptor, and requires only moderate intensity visible light. It has been discovered that a cross-linking reaction may occur in the absence of a photoactivatable metal-ligand complex. Such formulations require extended curing time, for example at least two hours, and potentially up to about 24 hours. In this way, compositions of the present disclosure, with or without a metal ligand complex, may form crosslinks in the absence of light. Thus, the methods disclosed herein may be practiced without irradiating the injected composition with light, such methods require a curing time of at least two hours, and may not be fully crosslinked for about 24 hours.

As used herein the term “electron acceptor” refers to a chemical entity that accepts electron transferred to it and so refers to an easily reduced molecule (or oxidizing agent) with a redox potential sufficiently positive to facilitate the cross-linking reaction. A range of electron acceptors will be suitable. In an embodiment, the electron acceptor is a peracid, a cobalt complex, a cerium (IV) complex, or an organic acid. Typically, the electron acceptor is a persulfate, periodate, perbromate or perchlorate compound, vitamin B12, Co(III) (NH₃)ₛCI²⁺, cerium (IV) sulphate dehydrate, ammonium cerium (IV) nitrate, oxalic acid or EDTA. An exemplary reaction is shown below:

$${S}_{2} {O}_{8}^{2 -} + 2 {H}^{+} + 2 {e}^{-} \text{=>} 2 {HSO}_{{4}^{-}}$$

is 2.1 V, as compared to 1.8 V for hydrogen peroxide (H₂O₂). This potential is higher than the redox potential for the permanganate anion (MnO₄⁻) at 1.7 V, but slightly lower than that of ozone at 2.2 V.

The coatings described herein may include one or more phenol-enriched polymers. The term “phenol enriched” as applied to a polymer material herein (e.g. collagen, gelatin, or a collagen peptide composition) means that the polymer material has been chemically modified to increase the number of phenolic groups in the polymer material. Thus, “phenol enriched collagen” refers to collagen that has been chemically modified to increase the number of phenolic groups (e.g. tyrosine groups) in the collagen, “phenol enriched gelatin” refers to gelatin that has been chemically modified to increase the number of phenolic groups in the gelatin, and “phenol enriched collagen peptide composition” refers to a collagen peptide composition that has been chemically modified to increase the number of phenolic groups in the collagen peptide composition. In some aspects, the phenolic groups are tyrosine groups, which can be added for example using a known Bolton Hunter reagent. In some aspects, the phenol enriched polymer material (e.g. collagen, gelatin, or collagen peptide composition) will have a P/G value of at least about 7, and in certain forms in the range of about 7 to about 35, or in the range of about 15 to about 30, or in the range of about 18 to about 25, where the P/G value is the number of moles of phenol groups per mole of polymer in the polymer material. The P/G value for a polymer material can be determined using standard techniques, including for example using an absorbance assay at a wavelength of 280 nm. Moderate P/G ranges for the phenol enriched polymer, as recited above, are preferred in some aspects, as modification to higher P/G values has been found to decrease the solubility of the material in aqueous media (see e.g. Example 1 below for phenol enriched gelatin).

Applicants have discovered that coatings as described herein provide a lubricious outer surface that upon implantation prevents adhesion of the coated material to surrounding soft tissues. In accordance with certain embodiments, the coating may contain one or more additives that alter the performance of the coating. Suitable additives for use herein may be included to improve lubricity, improve the aesthetics of the coating, reduce inflammation, and/or other beneficial changes. It is within the scope of the present disclosure to provide a coating having one or more additives, which may provide similar or different advantages. Thus, in certain embodiments, the present disclosure provides coating materials including additives for increasing the lubricity of the coating, such additives include but are not limited to: hyaluronic acid, sodium hyaluronate, and/or chondroitin sulfate. In some forms one or more hydrophilic additives may be included, suitable hydrophilic additives include sugars, for example fructose. Hydrophilic additives may cause the coating to form a more robust layer. In some forms, one or more additives may be included, which contribute additional resistance to adhesion, for example zwitterionic polymers.

It is also within the scope of the present disclosure to include one or more synthetic polymers in the coating layer. Such synthetic polymers may allow fine-tuning of the coating layer properties. Exemplary synthetic polymers include phenol-containing polymers, such as polyacrylamide and/or polyacrylic acid. Alternative embodiments may include a biodegradable polymer additive such as one or more of: polyethylene glycol, polyvinyl alcohol, polylactic acid, polyglycolic acid, polylactic-co-glycolic acid, and/or polyglycerol sebacate. In certain embodiments, the polymer is phenol-enriched by using Bolton-Hunter reagent or other suitable reaction.

The present disclosure provides methods of making an implantable medical graft. In some forms, the present disclosure provides methods including the step of applying a coating layer to a substrate as described herein. Such applying can be achieved in any suitable fashion, for example spraying, brushing, soaking, rolling, or any other suitable technique. After the coating is applied to the substrate, the resulting construct is covalently cross-linked to form a cross-linked coating layer. In accordance with some methods of the present disclosure, covalent crosslinks may be formed by irradiating a coated substrate. As disclosure herein, the present disclosure provides coating materials that form a diphenolic crosslinked polymer hydrogel under moderate intensity visible light. In certain embodiments, irradiation may be performed using white light, for example 450 nm nominal wavelength light. It is within the scope of the present disclosure to provide materials which form covalent crosslinks without direct irradiation as described above. For example, in some forms a coating layer may be applied to a substrate and allowed to cure with or without exposure to light. Curing of the coated substrate may take 2-48 hours, preferably 3-24 hours. In some forms a coated substrate is allowed to cure for at least 8 hours, at least 12 hours, or at least 24 hours. Such methods which do not require direct irradiation, may utilize a coating layer without a catalyst or comprising a catalyst other than a metal-ligand complex, for example riboflavin. In accordance with some forms, the present disclosure provides methods including drying a coated substrate. Such drying may occur simultaneously with said irradiating or may occur after said irradiating. Any suitable drying method may be utilized, for example lyophilization, e.g. freeze-drying.

In some forms, the photocured coating may include a combination of collagen and phenol enriched collagen, a combination of gelatin and phenol enriched gelatin, or a combination of a collagen peptide composition and a phenol enriched collagen peptide composition. In each case, the dry weight ratio of the parent polymeric material and its phenol enriched counterpart can be in the range of about 1:10 to about 10:1, or about 1:5 to about 5:1, or in some forms about 1:5 to about 1:2. Mixtures of two or more of collagen, gelatin, and a collagen peptide composition (each in its native form without phenol enrichment or as a phenol enriched polymeric material) can also be used.

In addition or alternatively, prior to photocuring the coating comprises a sterile liquid preparation that includes collagen, phenol enriched collagen, gelatin, phenol enriched gelatin, a collagen peptide composition, or a phenol enriched collagen peptide composition, or any mixture of two or more thereof, can exhibit the property of not gelling at 20° C., for example exhibiting no thermoreversible gelation activity upon cooling, or having a thermoreversible gelation temperature below 20° C., or below 15° C. In some forms, the sterile liquid preparation comprises gelatin, phenol enriched gelatin, or a mixture thereof, and the liquid preparation also includes an agent that inhibits the thermoreversible gelling of the gelatin (when present) and of the phenol enriched gelatin (when present). Urea is a preferred agent that inhibits this thermoreversible gelling, and can be used for example at a concentration in the range of about 1 molar to 5 molar in the liquid preparation, more typically about 3 molar to about 4.5 molar, and in some forms about 3.8 molar to about 4.5 molar. In other forms, the sterile liquid preparation includes a collagen peptide composition and/or a phenol enriched collagen peptide composition, that has an average molecular weight (Mw) below about 20,000 kilodaltons, more preferably below about 15,000 kilodaltons, and typically in the range of about 2,000 to about 12,000 kilodaltons. In these forms, the collagen peptide composition can exhibit no thermoreversible gelation activity upon cooling to 20° C. (or in some typical forms at any temperature), allowing the liquid preparation to remain a liquid at a temperature of 20° C., or at a temperature of 15° C. It will be understood that the liquid preparation may also remain a liquid at temperatures below these specified temperatures, and in general may remain a liquid throughout a temperature range expected to encompass room temperature storage and normal use temperatures, for example in the range of about 20° C. to about 37° C.

The sterile liquid preparation can include the polymer(s) containing phenolic groups in any suitable concentration. In some forms, the total concentration of the polymer(s) present in the sterile liquid preparation will be in the range of about 1% to about 40% weight/volume, more typically about 10% to about 40% weight/volume. In certain preferred forms, the sterile liquid preparation will include collagen, phenol enriched collagen, gelatin, phenol enriched gelatin, a collagen peptide composition, a phenol enriched collagen peptide composition, or any combination thereof, at a concentration in the range of about 20% to about 35% weight/volume, or in the range of about 25% to about 35% weight/volume. In such forms, the sterile liquid preparation, and coatings prepared using it, can be a flowable viscous liquid, for example having a viscosity at 20° C. of greater than about 300 centipoise, or greater than about 500 centipoise, and typically in the range of about 500 to about 20000 centipoise or in the range of about 1000 to about 10000 centipoise.

The sterile liquid preparation can include the photoactivatable catalyst such a photoactivtable metal ligand complex in a suitable amount to catalyze the formation of covalent crosslinks in the formation of the covalently crosslinked hydrogel by photocuring. Where a Ru(II) (bpy)3 complex such as [Ru(II) (bpy)3] Cl2 is used as the metal ligand complex, preferred sterile liquid preparations will include it at a concentration in the range of about 0.2 to about 2 mM, more desirably about 0.4 to about 1 mM. Where the electron acceptor to be mixed with the sterile liquid preparation is in dry powder form, the prepared photocurable liquid adhesive will have these same concentrations of the metal ligand complex. Where the electron acceptor is provided in a solution to be combined with the sterile liquid preparation, the concentration of the metal ligand complex in the prepared photocurable liquid adhesive will be reduced relative to that in the sterile liquid preparation. In some such forms, the volume of the sterile liquid preparation, the volume of the solution of electron acceptor, and the concentration of the metal ligand complex in the sterile liquid preparation, can be selected to provide a concentration of the metal ligand complex in the prepared photocurable coating material that is within the above-referenced concentration range values given for the sterile liquid preparation.

The sterile liquid preparation can have been terminally sterilized within the first chamber to render the liquid preparation sterile (e.g. using sterilizing radiation applied to a package containing the first container), but in some preferred forms the liquid preparation is sterilely prepared, for example including passage of the liquid preparation through a sterile filter, and then filled into the first chamber in a sterile filling operation. Such sterilely-filled liquid preparations in the first chamber can therefore be free from exposure to sterilizing radiation, and thus can be free from any degradation of the polymer(s) containing phenol groups caused by the sterilizing radiation. In some forms, the liquid preparation can be in a heated condition to reduce its viscosity during passage through the sterile filter. Also, in some forms, the first container having the first chamber containing the sterilely-filled liquid preparation can be sealed within a sterile barrier package under sterile conditions. Further, such sterile barrier package is preferably impermeable to visible light, as can be provided for example by a foil pouch package.

The implantable medical grafts described herein have broad application. In some aspects, inventive products will find use as precursor materials for the later formation of a variety of other medical products, or components thereof. Medical grafts and materials that are already commercially available can be modified in accordance with the present invention as well. In certain embodiments, inventive products are useful in procedures to replace, augment, support, repair, and/or otherwise suitably treat diseased or otherwise damaged or defective patient tissue. Some of the illustrative implantable medical grafts described herein will be useful, for example, in treating herniated tissue although coated materials and grafts as disclosed herein can be developed and used in many other medical contexts. In this regard, when used as a medical graft, the devices disclosed herein can be utilized in any procedure where the application of the graft to a bodily structure provides benefit to the patient. Illustratively, graft materials of the invention can be processed into various shapes and configurations, for example, into a variety of differently shaped urethral slings, surgical bolster or reinforcement materials (e.g., for use in tissue resection and similar procedures), wound products and other grafts and graft-like materials.

In certain embodiments, the coating is present in a uniform layer covering one or more surfaces of the underlying substrate. In accordance with certain preferred embodiments, the coating comprises a diphenolic crosslinked polymer hydrogel as described herein. In some forms, the substrate is generally sheet-form having a first surface and a second surface. In certain embodiments, the coating is present on the first surface while the second surface is free of coating. In some forms, a coating is present on both the first a second surfaces. The coating can be present in a variety of forms, for example in certain embodiments the coating is patterned on the surface of the substrate. The coating may be present in any suitable pattern, for example lines, cross-hatching, dots, or dots. Thus, in some forms a surface may have one or more coated portions and one or more uncoated portions. Such patterns may be advantageous, for example, to allow portions of the substrate to contact patient tissue, or to impart lubricous properties to only a portion of the substrate. As disclosed, herein patterned coating may be deployed to encourage directional folding of an implantable medical graft.

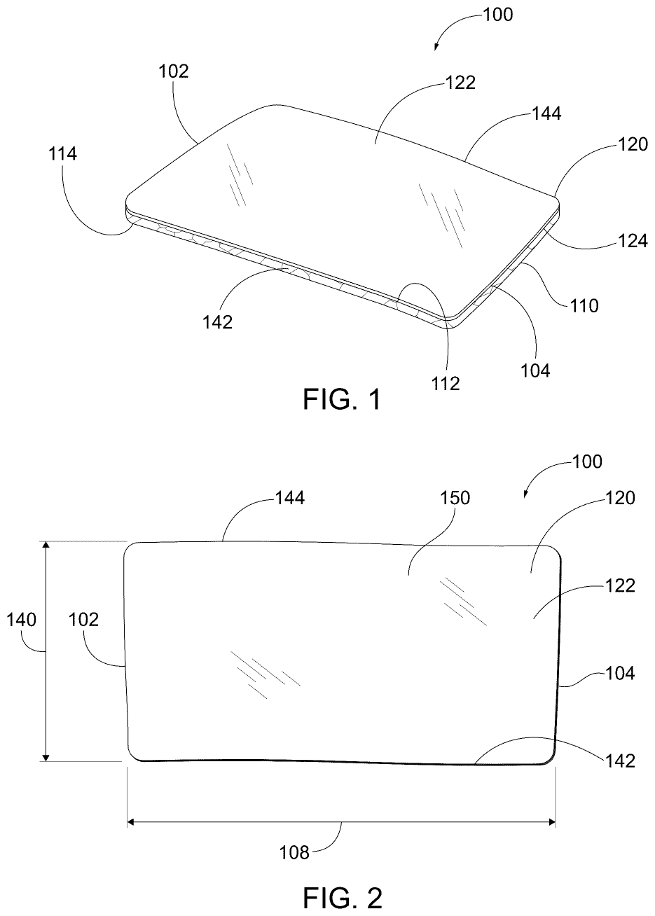

With reference now to the embodiment depicted in FIGS. 1 and 2, shown is one embodiment of an implantable medical graft 100. In the illustrated embodiment, the medical graft comprises a substrate 110 and at least a first coating 120. The coating comprises a substantially uniform layer disposed on a first face 112 of the substrate. A second face 114 of the substrate is free of the coating material. As shown, the coating is layered on at least one surface of the substrate. A first face 122 of the coating comprises an outer surface of the medical graft material. A second face 124 of the coating is contained within the medical graft material contacting and adhered to the first face of the substrate. Thus, the substrate comprises exposed portions and covered portions. In the illustrated embodiment, the substrate and the coating are present in substantially sheet form. As shown, the medical graft material has a length 108 extending from a first end 102 to a second end 104, and a width 140 extending from a first side 142 to a second side 144. In the illustrated embodiment, the coating comprises substantially all of the first face 150 of the medical graft material, extending the full length and width of the medical graft material.

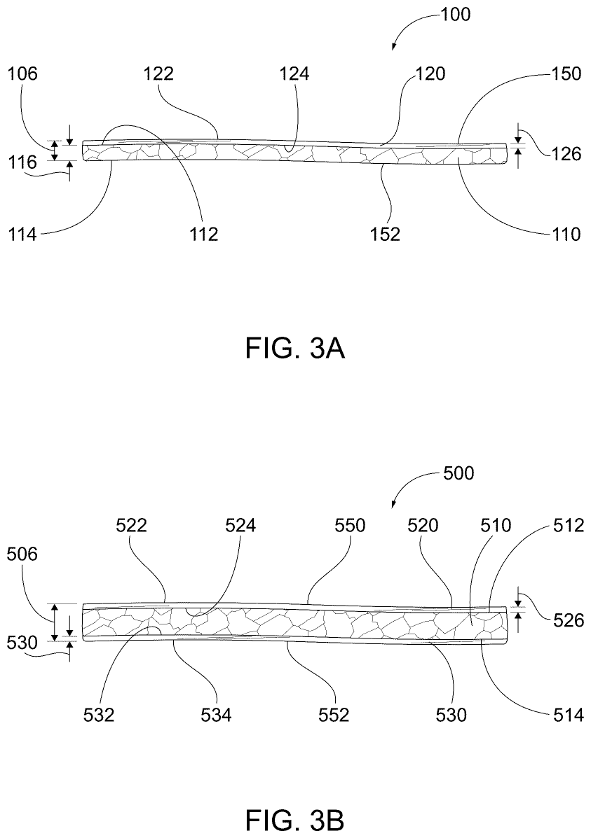

FIG. 3a is a side view of the medical graft material shown in FIGS. 1 and 2. Substrate 110 has a thickness 116 between first face 112 and second face 114. Coating 120 has a thickness 126 between first face 122 and second face 124. In the illustrated embodiment, the substrate thickness is greater than the thickness of the coating; however, it is within the scope of the disclosure to provide a coating thickness that is greater than the thickness of the substrate. As shown, medical graft material 100 has a thickness 106 between first face 150 and second face 152, the thickness of the medical graft includes the total thickness of the coating layer and the substrate.

FIG. 3b is a side view of one embodiment of a medical graft material 500 as described herein having a first coating layer 520 disposed on a first face 512 of substrate 510 and a second coating layer 530 disposed on a second face 514 of substrate 510. Substrate 510 has a thickness 516 between first face 512 and second face 514. First coating layer 520 has a thickness 526 between first face 522 and second face 524. Second coating later 530 has a thickness 536 between first face 532 and second face 534. In the illustrated embodiment, the substrate thickness is greater than the thickness of the coating layers; however, it is within the scope of the disclosure to provide a coating thickness that is greater than the thickness of the substrate. As shown, medical graft material 500 has a thickness 506 between first face 550 and second face 552, the thickness of the medical graft includes the total thickness of the coating layers and the substrate.

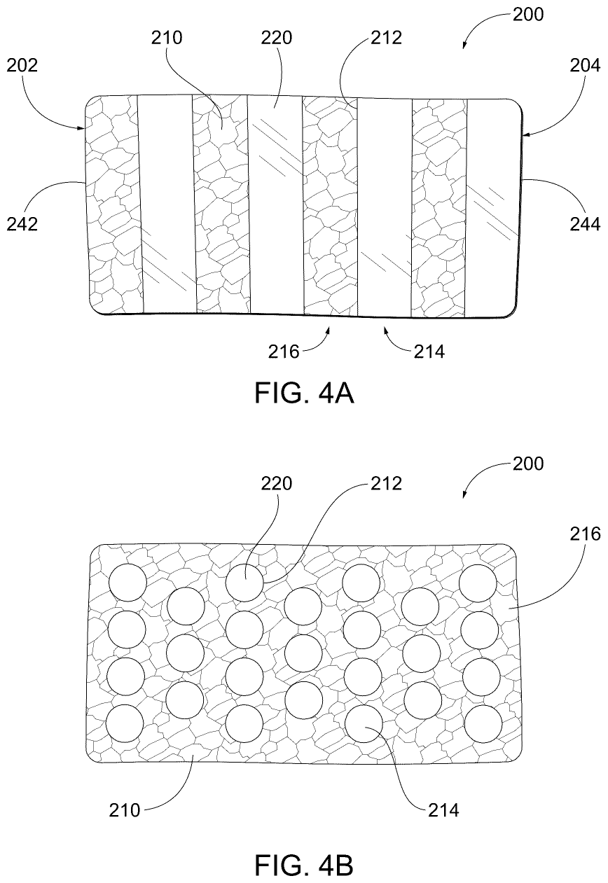

With reference now to FIGS. 4a and 4b, medical graft materials 200 as disclosed herein may include patterned coating layers 220 on one or more surface 212 of substrate 210. The substrate may include coated portions 214 and uncoated portions 216. The embodiment shown in FIG. 4a includes coated portions extending in striped pattern from first side 242 to second side 244. The illustrated embodiment shows four coated portions; however, it is within the scope of the disclosure to provide any number of coated portions, for example, one to twenty coated portions. First end 202 is uncoated and second end 204 is coated. In certain embodiments first end and second end are uncoated, in other embodiments first end and second end are coated. The embodiments shown in FIG. 4b includes coated portions in a dotted pattern across the surface of the substrate. In some forms, a medical graft material as disclosed herein may include various patterns, including striped portions and dots or other shaped coated portions.

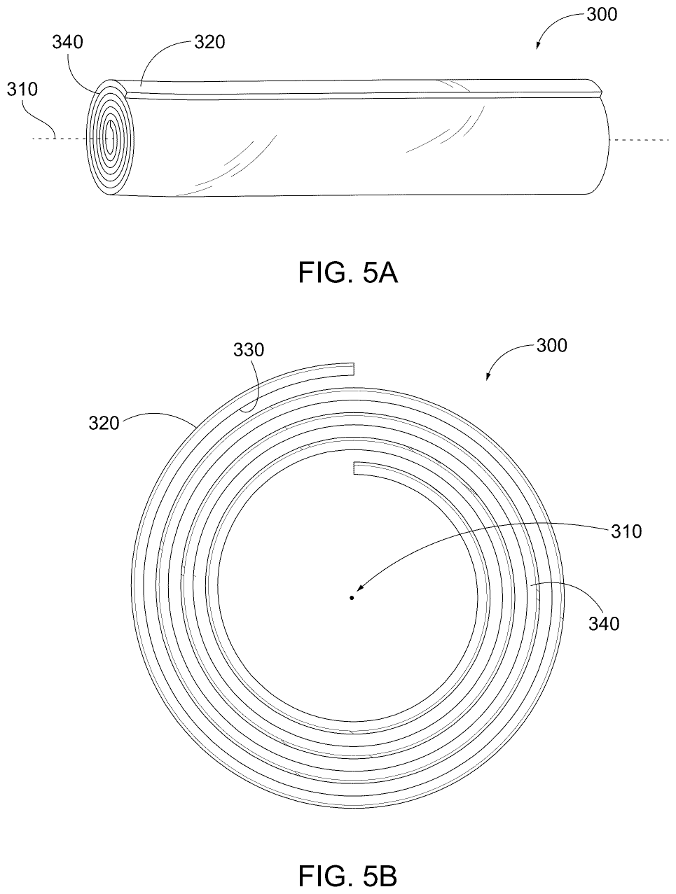

FIGS. 5a and 5b shows one embodiment of a medical graft material as disclosed herein, wherein the medical graft material 300 is curled about central axis 310. In the illustrated embodiment, first face 320 of the medical graft material adopts a convex configuration facing away from the central axis and second face 330 of the medical graft material adopts a concave configuration facing toward the central axis. The curled medical graft structure forms a generally cylindrical configuration with the outer surface comprising the coating layer. In accordance with some forms, the ends of the curled medical graft material, such as first end 340 comprise exposed end portions of the substrate material in a spiral configuration. FIG. 5b shows an end view of first end 340.

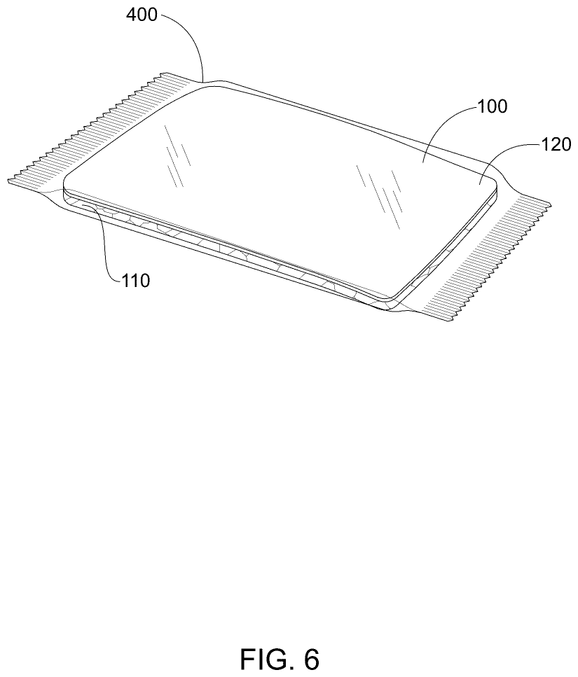

FIG. 6 shows one embodiment of a medical graft material as disclosed herein. In the illustrated embodiment, medical graft material 100 is contained within sterile package 400. In the illustrated embodiment, a medical graft material substantially similar to that shown in FIG. 1 is illustrated comprising substrate 110 and coating layer 120. It is within the scope of the disclosure to provide any of the medical graft materials described herein within a sterile medical package. In certain embodiments, the medical graft materials disclosed herein, including crosslinked coating layers, can be provided in sterile packaging suitable for medical materials and devices. Sterilization may be achieved, for example, by irradiation, ethylene oxide gas, or any other suitable sterilization technique, and the materials and other properties of the medical packaging will be selected accordingly. In accordance with some forms, the medical packaging selected may be opaque to prevent additional exposure of the medical graft material to light.

As disclosed herein, in certain embodiments the substrate may comprise a collagenous extracellular matrix material. It is also within the scope of the current disclosure to provide a substrate comprising other suitable materials. Suitable substrate materials are biocompatible and known to be implantable into human patients. For example, in some forms the substrate material comprises a surgical mesh. The substrate may comprise a synthetic material. Suitable synthetic materials include non-bioresorbable or bioresorbable synthetic polymer materials such as polytetrofluroethylene (PTFE, e.g. GORE-TEX material), nylon, polypropylene, polyurethane, silicone, DACRON polymer, polyglycolic acid (PGA), polylactic acid (PLA), polycaprolactone, or others. In some forms, the substrate material may include a collagenous extracellular matrix material and a synthetic polymer material. For example, a synthetic polymer material may be used to stitch layers of collagenous extracellular matrix materials together, or to reinforce one or more layers of collagenous extracellular matrix material. In certain embodiments, a synthetic polymer mesh may be present alongside, or between layers of collagenous extracellular matrix materials.

In some forms, the coating material as described herein may be present in a uniform layer over substantially all of a coated a face of the substrate material. In other embodiments, the coating is patterned unto the substrate face such that the face of the substrate material has coated portions and uncoated portions. The coating material may be applied in any suitable pattern, for example, linear segments extending from one end of the graft to the other. In some forms, the coating is present in shaped sections, such as one or more circular or polygonal shaped coated portions on the surface of the substrate. In accordance with some forms, the coating pattern may be selected to optimize surface characteristics, such as lubricity of the material. In some embodiments, the coating pattern may be selected to encourage the medical graft material to curl or fold in a predetermined manner. For example, it has been discovered that linear coating patterns, such as a stripe pattern, may be used to encourage curling of the graft material along the linear direction of the coating.

Thus, in accordance with some forms, the present disclosure provides for a sheet form material having a stitching material present on a surface layer. Such stitching material may be present, for example, to bond together a multilaminate material. In certain embodiments, a stitching material may be used to reinforce a sheet form material having one or more layers. Such stitching material may be present in a stitching pattern around the periphery of a sheet form material. In certain embodiments the stitching material passes through a thickness of the sheet form medical material (e.g. passing through multiple layers when present) such that the stitching material is present on opposite faces of the sheet form material. The stitching material may comprise either a synthetic or natural (e.g., collagenous) material. In certain embodiments the stitching material comprises a synthetic polymeric material. Suitable synthetic materials include non-bioresorbable or bioresorbable synthetic polymer materials such as polytetrofluroethylene (PTFE, e.g. GORE-TEX material), nylon, polypropylene, polyurethane, silicone, DACRON polymer, polyglycolic acid (PGA), polylactic acid (PLA), polycaprolactone, or others. In accordance with some forms, the stitching material is bioresorbable such that the stitching material degrades and is resorbed by the body as the medical material remodels. However, a non-resorbable synthetic stitching material, such as a suture made of cross-linked collagen or a non-resorbable synthetic polymer may be used, particularly if there is a desire or need to enhance the strength of the medical material and/or provide continued reinforcement to compliment the remodeled tissue. In this way, the present disclosure provides for a stitched material having a coating as described herein which lies over all or a portion of the individual stitches. In this way an otherwise textured surface, due to the presence of the stitches, may be covered by a coating layer and present a smooth outer layer.

It is also within the scope of the present disclosure to provide a medical graft material comprising a coating on both faces of a sheet-form substrate material. For example, a substrate material may be provided having a first face opposing a second face, and a first coating layer is deposited on the first face and a second coating layer is deposited on the second face. As disclosed above the coating layers may coat the entire face or only a coated portion leaving uncoated potions. The two faces may be coated in the same fashion, e.g. each having a uniform or patterned coating. It has been discovered that sheet form graft materials that are uniformly coated on both sides resist curling. Alternatively, the two faces may be coated differently, for example, a first surface may have a uniform coating while the second face has a patterned coating.

In certain embodiments, the present disclosure provides medical grafts having a curled configuration in a relaxed state. In such embodiments, the medical graft is curled about a central axis such that one face of the graft adopts a convex configuration facing towards the central axis and a second face of the graft adopts a convex configuration facing away from the central axis. In certain embodiments, the convex face of the medical graft material includes a coating as described herein. Such curled graft materials may be useful, for example for implantation around or within a ligament, peripheral nerves, and/or vessel or other tubular, cylindrical, or elongate shaped structure.

Medical graft devices as described herein provide a lubricious surface for reducing friction and adhesion with adjacent tissue upon implantation. For example, a hernia repair graft may be provided comprising a medical graft material as described herein. In use, a coated side of the device is implanted oriented facing towards the internal organs (e.g. intestines) so as to reduce friction between the organs and the implanted graft material thereby reducing the formation of tissue adhesions with the graft material.

Any or all of the components described herein can be provided in a sterile pack for providing necessary parts, or a variety of parts, to a surgeon. For example, one or more crosslinked medical grafts may be provided in a single sterile package or kit. A surgeon can choose the sizes or types of medical graft he or she wishes to use during surgery. For example, a surgeon may modify the size and/or shape of the medical graft prior to implantation. Alternatively, sterile kits containing predetermined sizes or types of components may be provided. Packages or kits of the components described herein can include additional devices or tools which may be useful in the particular medical procedure being performed.

### EXAMPLE 1

### Phenol Enriched Gelatin Prepared with Bolton Hunter Reagent

Nippi MediGelatin (derived from porcine skin; Mw approximately 100 kilodaltons) was dissolved in high purity water with 6.18 g/L boric acid, 9.54 g/L sodium borate, and 4.38 g/L sodium chloride at 30° C. and 60° C. at a concentration of 10 g/L in a 1 L reaction volume (n=1). 200 mL aliquots of this solution were extracted into 500 mL Erlenmeyer flasks and combined with 3.5 mL of a Bolton Hunter reagent/DMSO solution (Bolton Hunter reagent=N-succinimidyl-3-[4-hydroxyphenyl]propionate). The Bolton Hunter/DMSO solution was prepared at a concentration such that the final concentration of Bolton Hunter reagent in the gelatin solution ranged from 0.2 to 5 g/L. The mixture was reacted at 40° C. in a shaken incubator for two hours. The solution was dialyzed, dried, and analyzed for phenol content using absorbance at 280 nm. Unless noted otherwise, groups had a replicate size of n=3. Each group was evaluated for normality and compared across groups for equal variance. Groups were then compared for statistical difference using a one-way ANOVA (α=0.05) and Tukey post-hoc tests.

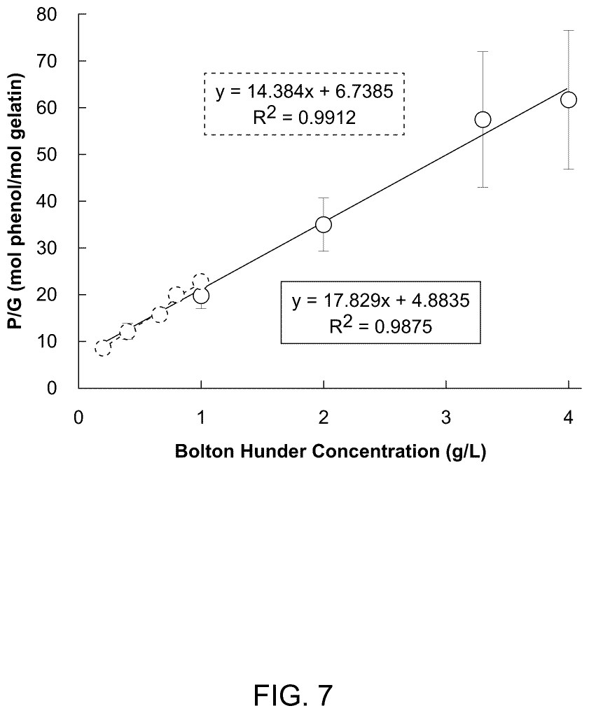

The results are summarized in FIG. 7. P/G values (mole phenol/mole gelatin) for the modified materials ranged from just under 10 to about 60 (the unmodified gelatin had a P/G value of about 3). At all concentrations at and below 1 g/L Bolton Hunter, the resulting phenol enriched gelatin was soluble in PBS. The relationship between Bolton Hunter concentration and extent of phenol enrichment was linear, with a 10% change in Bolton Hunter concentration causing a ˜6% change in the molar ratio of phenol to gelatin. At Bolton Hunter concentrations at 2 g/L and higher, the relationship became less linear and the measurements had higher standard deviation. In addition, the modified gelatin became less soluble in PBS, with protein modified at 5 g/L becoming completely insoluble. These results indicate that a severe excess of Bolton Hunter reagent and a resulting very high P/G value for the phenol enriched protein can decrease the solubility of the modified protein in an aqueous medium.

### EXAMPLE 2

### Phenol Enriched Gelatin Prepared by Carbodiimide Reaction

Gelatin was modified to include additional phenol groups using EDC (1-ethyl-3-(3-dimethylaminopropyl)carbodiimide hydrochloride), NHS (N-hydroxysuccinimide), and HPPA (3-(4-Hydroxyphenyl) propionic acid). A precipitate was first prepared using a 5:2:1 ratio of NHS: HDC: HPPA at concentrations of 325 mM NHS, 130 mM EDC and 65 mM HPPA. First, HPPA was solubilized in a 0.1M MES, 0.9% Sodium Chloride, pH 4.7 buffer on a stir plate at 200 rpm. Once the HPPA was dissolved, the EDC and NHS were added to the solution. After 15-20 minutes a precipitate began to form. The solution was allowed to react for 2 to 4 hours and then vacuum filtered. Following double filtration of the solution, the precipitate captured on the filter paper was allowed to dry in a fume hood for at least 24 hours. Once the precipitate was dry, it was utilized as solubilized in DMSO in 4× mass in place of the Bolton-Hunter Reagent to add phenolic groups to the gelatin.

Modified gelatin prepared using the precipitate in place of Bolton-Hunter reagent was formulated into photocurable adhesive compositions using a phosphate buffered saline medium, bipyridyl) ruthenium (II) chloride hexahydrate and sodium persulfate. Photocurable adhesive formulations of having 5:1, 1:1 and 0:1 ratios of unmodified gelatin to modified gelatin were prepared. The thus prepared photocurable adhesive demonstrated the ability to cure under visible light.

The use of the terms “a” and “an” and “the” and similar referents in the context of describing the invention especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Embodiments

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.
1. An implantable medical graft product, comprising:
   a package;
   an implantable medical graft received in the package in sterile condition, the medical graft including:
      a substrate comprising a collagenous extracellular matrix material;
      a coating disposed on a first surface of said substrate, said coating comprising a covalently crosslinked matrix protein hydrogel having diphenolic crosslinks between individual matrix protein molecules.
2. The implantable medical graft product of embodiment 1, wherein said coating comprises hyaluronic acid or a salt thereof.
3. The implantable medical graft of any of the preceding embodiments, wherein said coating comprises sodium hyaluronate.
4. The implantable medical graft of any of the preceding embodiments, wherein said coating comprises chondroitin sulfate.
5. The implantable medical graft of any of the preceding embodiments, wherein said coating comprises one or more added sugars.
6. The implantable medical graft of any of the preceding embodiments, wherein said collagenous extracellular matrix material is a membranous extracellular matrix sheet material comprising submucosa, dura mater, pericardium, basement membrane, or dermal collagen.
7. The implantable medical graft of any of the preceding embodiments, wherein the first surface is on a first face of said substrate, and wherein a second face of said substrate is not coated.
8. The implantable medical graft of any of the preceding embodiments, wherein the first surface is on a first face of said substrate, and wherein a second face of said substrate has disposed thereon a coating comprising a covalently crosslinked matrix protein hydrogel having diphenolic crosslinks between individual matrix protein molecules.
9. The implantable medical graft of any of the preceding embodiments, wherein said medical device is lyophilized.
10. The implantable medical graft of any of the preceding embodiments, comprising: one or more openings in said medical graft, said openings extending through said coating and said substrate.
11. The implantable medical graft of any of the preceding embodiments, wherein said coating covers substantially all of a first face of the substrate.
12. The implantable medical graft of any of the preceding embodiments, wherein substrate has a first face presenting a first face surface, and wherein first portions of the first face surface have said coating disposed thereon and second portions of the first face surface do not have said coating disposed thereon.
13. The implantable medical graft of any of the preceding embodiments, wherein in a relaxed state said implantable medical graft is curled about a central axis such that a first face of the medical graft adopts a convex configuration facing away from said central axis, and wherein said first surface is on said first face.
14. The implantable medical graft of embodiment 13, comprising an opening extending the length of the medical graft along said central axis from a first end to a second end of the medical graft.
15. The implantable medical graft of any of the preceding embodiments, wherein said crosslinked matrix protein hydrogel comprises gelatin.
16. The implantable medical graft of any of the preceding embodiments, wherein said coating is dehydrated.
17. The implantable medical graft of any of the preceding embodiments, comprising: a stitching material on said first surface, wherein said coating is disposed over said stitching material.
18. A method for making an implantable medical graft product, said method comprising: covalently crosslinking a matrix protein coating material disposed on a first surface of a collagenous extracellular matrix substrate material, wherein said covalently crosslinking is effective to form a covalently crosslinked matrix protein hydrogel coating on the substrate, the matrix protein hydrogel being in hydrated condition and having diphenolic cross-links between matrix protein molecules of the matrix protein hydrogel; and sterilely packaging the substrate having the covalently crosslinked matrix protein hydrogel thereon.
19. The method of embodiment 18, wherein the coating comprises hyaluronic acid or a salt thereof.
20. The method of any one of embodiments 18 or 19, wherein the coating comprises sodium hyaluronate.
21. The method of any one of embodiments 18 through 20, wherein the coating comprises chondroitin sulfate.
22. The method of any one of embodiments 18 through 21, wherein the coating comprises one or more added sugars.
23. The method of any one of embodiments 18 through 22, wherein the collagenous extracellular matrix substrate material is a membranous extracellular matrix material comprising submucosa, dura mater, pericardium, basement membrane, or dermal collagen.
24. The method of any one of embodiments 18 through 23, wherein the first surface is on a first face of the substrate, and wherein a second face of the substrate is not coated.
25. The method of any one of embodiments 18 through 24, wherein the first surface is on a first face of the substrate, and wherein a second face of the substrate has disposed thereon a coating comprising a covalently crosslinked matrix protein hydrogel having diphenolic crosslinks between individual matrix protein molecules.
26. The method of any one of embodiments 18 through 25, comprising: drying the covalently crosslinked matrix protein hydrogel.
27. The method of embodiment 26, comprising:
   sterilely packaging the medical graft product after said drying.
28. The method of embodiment 26, wherein the said drying comprises lyophilizing.
29. The method of any one of embodiments 18 through 28, comprising:
   forming one or more openings in the crosslinked medical graft, the openings extending through the coating and the substrate.
30. The method of any one of embodiments 18 through 29, wherein the coating covers substantially all of a face of the collagenous extracellular matrix material.
31. The method of any one of embodiments 18 through 30, wherein a first face of the substrate comprises coated portions and uncoated portions.
32. The method of any one of embodiments 18 through 31, wherein in a relaxed state the crosslinked medical graft is curled about a central axis such that a first face adopts a convex configuration facing away from the central axis, and wherein said first surface is on said first face.
33. The method of any one of embodiments 30 through 32, comprising an opening extending the length of the medical graft material along said central axis from a first end to a second end.
34. The method of any one of embodiments 18 through 33, wherein the matrix protein comprises gelatin.
35. A method of treating a patient, the method comprising:
   implanting a crosslinked medical graft comprising a collagenous extracellular matrix material substrate and a coating disposed on at least a first face of the substrate, the coating comprising a covalently crosslinked matrix protein hydrogel having diphenolic crosslinks between individual matrix proteins molecules.
36. The method of embodiment 32, comprising:
   removing the crosslinked medical graft from a sterile package.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. An implantable medical graft product, comprising:
a package;
an implantable medical graft received in the package in sterile condition, the medical graft including:
a substrate comprising a collagenous extracellular matrix material;
a coating disposed on a first surface of said substrate, said coating comprising a covalently crosslinked matrix protein hydrogel having diphenolic crosslinks between individual matrix protein molecules.

2. The implantable medical graft product of claim 1, wherein said coating comprises hyaluronic acid or a salt thereof, sodium hyaluronate, chondroitin sulfate, and/or one or more added sugars.

3-6. (canceled)

7. The implantable medical graft of claim 1, wherein the first surface is on a first face of said substrate, and wherein a second face of said substrate is not coated.

8. The implantable medical graft of claim 1, wherein the first surface is on a first face of said substrate, and wherein a second face of said substrate has disposed thereon a coating comprising a covalently crosslinked matrix protein hydrogel having diphenolic crosslinks between individual matrix protein molecules.

9. The implantable medical graft of claim 1, wherein said medical device is lyophilized.

10. (canceled)

11. The implantable medical graft of claim 1, wherein said coating covers substantially all of a first face of the substrate.

12. The implantable medical graft of claim 1, wherein substrate has a first face presenting a first face surface, and wherein first portions of the first face surface have said coating disposed thereon and second portions of the first face surface do not have said coating disposed thereon.

13. The implantable medical graft of claim 1, wherein in a relaxed state said implantable medical graft is curled about a central axis such that a first face of the medical graft adopts a convex configuration facing away from said central axis, and wherein said first surface is on said first face.

14. The implantable medical graft of claim 13, comprising an opening extending the length of the medical graft along said central axis from a first end to a second end of the medical graft.

15. The implantable medical graft of claim 1, wherein said crosslinked matrix protein hydrogel comprises gelatin.

16. (canceled)

17. The implantable medical graft of claim 1, comprising:
a stitching material on said first surface, wherein said coating is disposed over said stitching material.

18. A method for making an implantable medical graft product, said method comprising:
covalently crosslinking a matrix protein coating material disposed on a first surface of a collagenous extracellular matrix substrate material, wherein said covalently crosslinking is effective to form a covalently crosslinked matrix protein hydrogel coating on the substrate, the matrix protein hydrogel being in hydrated condition and having diphenolic cross-links between matrix protein molecules of the matrix protein hydrogel; and
sterilely packaging the substrate having the covalently crosslinked matrix protein hydrogel thereon.

19. The method of claim 18, wherein the coating comprises hyaluronic acid or a salt thereof, sodium hyaluronate, chondroitin sulfate, and/or one or more added sugars.

20-23. (canceled)

24. The method of claim 18, wherein the first surface is on a first face of the substrate, and wherein a second face of the substrate is not coated.

25. The method of claim 18, wherein the first surface is on a first face of the substrate, and wherein a second face of the substrate has disposed thereon a coating comprising a covalently crosslinked matrix protein hydrogel having diphenolic crosslinks between individual matrix protein molecules.

26. The method of claim 18, comprising:
drying the covalently crosslinked matrix protein hydrogel, optionally wherein the said drying comprises lyophilizing.

27. The method of claim 26, comprising:
sterilely packaging the medical graft product after said drying.

28. (canceled)

29. (canceled)

30. The method of claim 18, wherein the coating covers substantially all of a first face of the collagenous extracellular matrix material.

31. The method of claim 18, wherein a first face of the substrate comprises coated portions and uncoated portions.

32. The method of claim 18, wherein in a relaxed state the crosslinked medical graft is curled about a central axis such that a first face adopts a convex configuration facing away from the central axis, and wherein said first surface is on said first face.

33. The method of claim 30, comprising an opening extending the length of the medical graft material along said central axis from a first end to a second end.

34. The method of claim 18, wherein the matrix protein comprises gelatin.

35. A method of treating a patient, the method comprising:
implanting a crosslinked medical graft comprising a collagenous extracellular matrix material substrate and a coating disposed on at least a first face of the substrate, the coating comprising a covalently crosslinked matrix protein hydrogel having diphenolic crosslinks between individual matrix proteins molecules.

36. The method of claim 32, comprising:
removing the crosslinked medical graft from a sterile package.
